(19) United States
(12) Patent Application Publication
(10) Pub. No.: US 2008/0206852 A1
(43) Pub. Date: Aug. 28, 2008

(54) SAMPLE PROCESSING APPARATUS

(75) Inventor: Yasuyuki Numajiri, Kawasaki-shi (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 12/033,511

(22) Filed: Feb. 19, 2008

(30) Foreign Application Priority Data
Feb. 22, 2007 (JP) 2007-042660

(51) Int. Cl. C12M 1/34; C12M 1/00

(52) U.S. Cl. 435/287.2; 435/287.3; 435/307.1

(57) ABSTRACT

The present invention provides a sample processing apparatus and a sample container. In a sample processing apparatus, only after a locking member provided on a sample container engages with a sample container receiving portion, the locking member is released. Thereby, a sample fluid is transferred from the sample container to the sample processing apparatus so that various analysis processes can be performed.

### BACKGROUND OF THE INVENTION

1. Field of the Invention

The present invention relates to a sample processing apparatus used for analyzing samples of blood, urine, stool, sputum, cerebrospinal fluid, tissues, and the like collected from human or animal subjects.

2. Description of the Related Art

In order to transfer a sample fluid which contains a sample or a substance obtained from a sample and which is stored in a sample container to a sample processing apparatus, when the sample container is not equipped with a plug, it is common that the sample fluid is drawn by suction with a pipette and dispensed into the sample processing apparatus. When the sample container is equipped with a plug, the plug is removed, and then similarly the sample fluid is drawn by suction with a pipette and dispensed into the sample processing apparatus. Alternatively, a hollow needle communicating with a syringe or a pump is allowed to penetrate the plug so that the hollow needle is brought into contact with the sample fluid, and then the sample fluid is drawn by suction and dispensed into the sample processing apparatus.

Japanese Patent Laid-Open No. 6-300670 discloses a method in which a through-chip having a hollow nozzle is pressed in a plug of a blood-collecting tube by a pressing unit, and suction or injection is performed by inserting a nozzle into the through-chip.

Japanese Patent Laid-Open No. 6-347466 discloses a method in which a plug is removed from a blood-collecting tube which has been subjected to centrifugal separation, then fibrin in serum is examined, and the serum which has passed the fibrin examination is dispensed into another container.

Furthermore, Japanese Patent Laid-Open No. 7-077527 discloses a method in which a blood-collecting tube sealed with a plug is turned upside down so that the plug is oriented downward, a hollow needle is allowed to penetrate the plug from the bottom, and by repeatedly increasing and decreasing the pressure, serum is transferred to a collecting container placed on the lower side.

Furthermore, Japanese Patent Laid-Open No. 2006-250860 discloses a cylindrical sample container from which a sample fluid is directly transferred to a reactor container, in which by applying a pressure to the inside of the sample container, a stopper for the sample fluid is allowed to slide to form a gap, and thereby the sample fluid is dripped into the reactor container.

As described above, although various methods have been proposed for transferring a sample fluid in a sample container to a sample processing apparatus, in any of these methods, there has been a possibility that the sample fluid may scatter to the outside of the sample container when the sample fluid is transferred to the outside of the sample container.

### SUMMARY OF THE INVENTION

The present invention provides a sample processing apparatus in which, when a sample fluid stored in a sample container is transferred to the sample processing apparatus, there is a lower possibility that the sample fluid may scatter to the outside of the sample container.

In an aspect of the present invention, a sample processing apparatus includes a sample container having a reservoir that stores a sample fluid, a fluid outlet port provided on the reservoir, and a locking member capable of opening and closing the fluid outlet port; a sample container receiving portion having an opening and engageable with the locking member; and an analysis processing portion that communicates with the opening. When the locking member of the sample container engages with the sample container receiving portion and the engaged locking member moves relative to the sample container, the fluid outlet port is opened, and the sample fluid stored in the reservoir moves through the opening to the analysis processing portion so that the sample fluid can be subjected to an analysis process.

In another aspect of the present invention, a sample container includes a reservoir that stores a sample fluid therein, a fluid outlet port provided on the reservoir, and a locking member that opens and closes the fluid outlet port. The sample container can be attached to and detached from a sample processing apparatus provided with a sample container receiving portion. When the locking member engages with the sample container receiving portion and the engaged locking member moves relative to the sample container, the fluid outlet port is opened.

According to the present invention, it is possible to reduce the possibility of scattering of the sample fluid to the outside of the sample container.

Further features of the present invention will become apparent from the following description of exemplary embodiments with reference to the attached drawings.

### BRIEF DESCRIPTION OF THE DRAWINGS

FIGS. 1A and 1B are schematic diagrams of a sample container according to a first embodiment of the present invention.

FIGS. 2A and 2B are schematic diagrams showing a part of a device to which a sample is transferred from the sample container according to the first embodiment and a part of a sample processing apparatus.

FIGS. 3A and 3B are schematic diagrams showing a state in which the sample container according to the first embodiment is connected to the device to which the sample is transferred, and the sample communicates with the device.

FIGS. 4A and 4B are schematic diagrams of a sample container according to a second embodiment of the present invention.

FIGS. 5A and 5B are schematic diagrams showing a part of a device to which a sample is transferred from the sample container according to the second embodiment and a part of a sample processing apparatus.

FIGS. 6A and 6B are schematic diagrams showing a state in which the sample container according to the second embodiment is connected to the device to which the sample is transferred, and the sample communicates with the device.

FIGS. 7A and 7B are schematic diagrams of a sample container according to a third embodiment of the present invention.

FIGS. 8A and 8B are schematic diagrams showing a part of a device to which a sample is transferred from the sample container according to the third embodiment and a part of a sample processing apparatus.

FIGS. 9A and 9B are schematic diagrams showing a state in which the sample container according to the third embodiment is connected to the device to which the sample is transferred, and the sample communicates with the device.

### DESCRIPTION OF THE EMBODIMENTS

### First Embodiment

A first embodiment of the present invention will be described in detail with reference to the drawings.

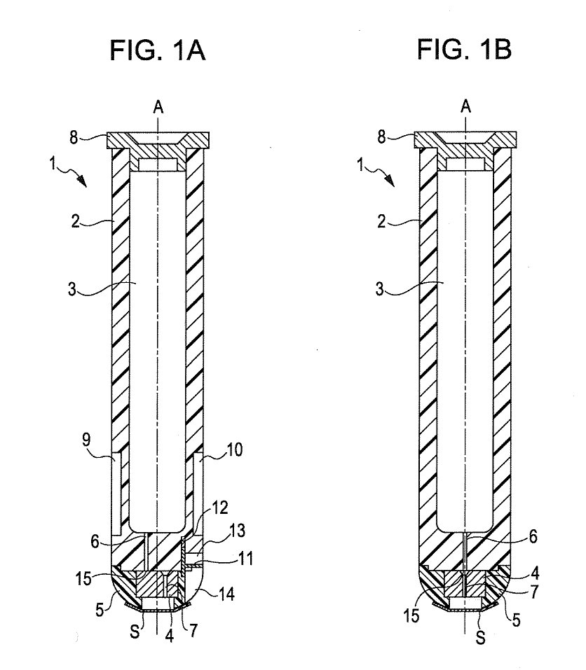

FIGS. 1A and 1B are schematic diagrams showing a sample container that can be used for a sample processing apparatus according to an embodiment of the present invention. FIG. 1A is a cross-sectional view taken along a plane including grooves 9 and 10 provided on the side face of a sample container 1 and a locking member 4. FIG. 1B is a cross-sectional view of the sample container taken along a plane rotated from the cross section of FIG. 1A by 90 degrees with respect to axis A.

Referring to FIGS. 1A and 1B, a main body 2 of the sample container 1 has therein a reservoir 3 that stores a sample of a collected fluid, such as blood. The sample container main body 2 includes on the bottom thereof a locking member 4 that can open and close a fluid outlet port 15 so as to prevent the sample fluid, such as blood, from scattering to the outside. The sample container main body 2 is composed of a synthetic resin, such as polystyrene, polycarbonate, or polymethyl methacrylate. The locking member 4 is pressed against the sample container main body 2 by a locking member retainer 5 held in a rotatable manner with respect to axis A. The locking member retainer 5 can rotate with respect to the sample container main body 2. The locking member 4 is restricted from rotating by the locking member retainer 5 and turns in response to turning of the locking member retainer 5. For this purpose, the locking member retainer 5 is composed of a synthetic resin having good slidability, such as nylon, polyacetal, polypropylene, or a fluorocarbon resin. The locking member 4 is composed of an elastomer, such as silicone rubber, nitrile rubber, fluororubber, ethylene-propylene rubber, butyl rubber, or a thermoplastic elastomer, in order to improve liquid tightness between the locking member 4 and the sample container main body 2. In this embodiment, the locking member 4 and the locking member retainer 5 cooperate to open and close the fluid outlet port 15 such that the sample fluid is prevented from scattering to the outside. Therefore, both components can be considered to constitute one locking member.

A channel 6, which has a cross-sectional area smaller than the area of the bottom surface of the reservoir 3, and the fluid outlet port 15 are disposed in a part of the sample container main body 2 between the reservoir 3 and the locking member 4. A channel 7 is disposed in the locking member 4. The locking member 4 can be turned with respect to axis A along with the locking member retainer 5, and by turning the locking member 4 with an appropriate angle, the channel 6 and the channel 7 can communicate with each other. A plug 8 is disposed on the upper part of the sample container main body 2, and two grooves 9 and 10 are provided on the side face of the sample container main body 2. Furthermore, a rotation stopper 11 serving as a movement prohibiting portion, which prohibits the locking member retainer 5 from turning, is provided on the lower part of the sample container main body 2. The rotation stopper 11 is pressed toward the locking member retainer 5 by a spring 12 and inserted into a recess provided in the lock member retainer 5. As long as the rotation stopper 11 is inserted into the recess, the locking member retainer 5 is prohibited from turning. A groove 13 and a groove 14 are disposed in the sample container main body 2 and the locking member retainer 5, respectively, so that the rotation stopper 11 can slide therein. Furthermore, a protective seal S, which prevents the air-exposed surface of the locking member 4 from being contaminated, may be attached to the locking member retainer 5 as shown in FIGS. 1A and 1B.

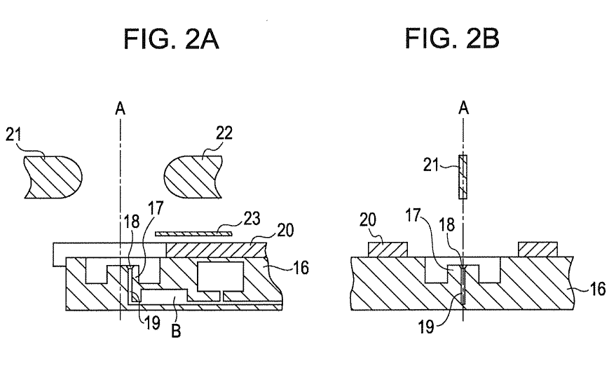

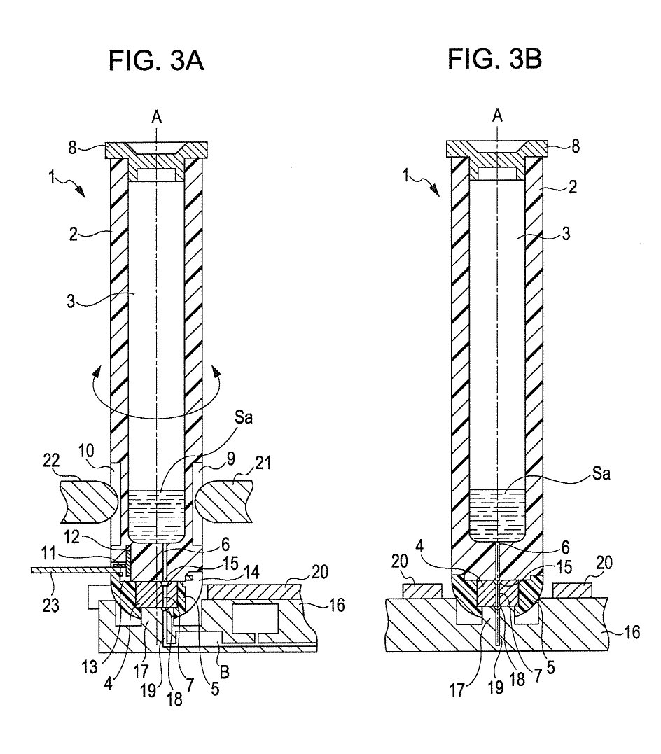

A method for transferring a sample fluid, such as blood, from the attachable/detachable sample container 1 to the sample processing apparatus according to this embodiment will be described below with reference to FIGS. 2A, 2B, 3A, and 3B. FIGS. 2A and 2B each shows a cross-sectional view of a micro-fluidic device, which is also referred to as micro-total analysis system (μ-TAS), as an example of the sample processing apparatus. FIG. 2A is a cross-sectional view taken along a plane including an opening 18 of the sample processing apparatus and a channel 19 communicating with the opening 18. FIG. 2B is a cross-sectional view of the micro-fluidic device taken along a plane rotated from the cross section of FIG. 2A by 90 degrees with respect to axis A.

In FIGS. 2A and 2B, reference numeral 16 represents a micro-fluidic device, to which the sample container 1 according to this embodiment can be connected. The sample container 1 can be connected in a detachable manner to the main body of the micro-fluidic device 16 through a sample container receiving portion 17. The opening 18, which can communicate with the channel 7 provided in the locking member 4, is disposed in the sample container receiving portion 17. The channel 19 connected to the opening 18 is also disposed in the sample container receiving portion 17. Furthermore, in the sample processing apparatus according to this embodiment, a device retainer 20 that fixes the micro-fluidic device 16, rotating plates 21 and 22, and a rotation stopper release bar 23 are disposed. The rotating plates 21 and 22 are inserted into the grooves 9 and 10 of the sample container main body 2, respectively, so that the sample container main body 2 is rotated with respect to the locking member retainer 5. The rotation stopper release bar 23 allows the rotation stopper 11 to slide along the groove 14 and releases the prohibition of movement of the locking member retainer 5 by the rotation stopper 11. In such a manner, a sample container may be formed so as to have a structure that prohibits the movement of the locking member using the rotation stopper 11, the spring 12, and the groove 14.

A method for transferring a sample fluid stored in the sample container using the sample processing apparatus according to this embodiment will be described in detail with reference to FIGS. 3A and 3B. Symbol Sa shown in FIGS. 3A and 3B represents a sample fluid stored in the reservoir 3. FIGS. 3A and 3B are schematic diagrams showing a state in which the sample container 1 according to this embodiment is connected to and communicates with the micro-fluidic device 16. FIG. 3A is a cross-sectional view taken along a plane including the opening 18 of the sample processing apparatus and the channel 19 communicating with the opening 18 as in FIG. 2A. FIG. 3B is a cross-sectional view taken along the plane similar to that of FIG. 2B. Axis A shown in FIGS. 3A and 3B corresponds to axis A shown in FIGS. 2A and 2B, and may be used below when describing the connection between the sample container and the micro-fluidic device in the present specification.

First, the protective seal S attached to the sample container 1 is removed. Then, from the upper side of the micro-fluidic device 16 fixed by the device retainer 20, the sample container 1 is connected to the sample container receiving portion 17 along axis A as an engaging axis between the sample container 1 and the micro-fluidic device 16 such that the groove 14 faces the rotation stopper release bar 23. In such a manner, as shown in FIGS. 3A and 3B, the opening 18 and the fluid outlet port 15 can be closely engaged with each other through the locking member 4. When the sample container 1 is engaged with the sample container receiving portion 17, the rotation stopper 11 is allowed to slide upward by the rotation stopper release bar 23, and the prohibition of movement of the locking member retainer 5 is released. As shown in FIGS. 3A and 3B, the sample container receiving portion 17 is configured so as to have a shape that allows the locking member 4 and the locking member retainer 5 to be engaged with each other. For example, with respect to a sample container receiving portion having an equilateral triangular cross section, a locking member retainer having an equilateral triangular recess so as to be engageable with the sample container receiving portion is provided on the sample container. Of course, the shapes of the sample container receiving portion and the locking member retainer are not particularly limited as long as they can be engaged with each other.

Next, the sample container 1 is pressed from the upper side by a mechanism (not shown) of the sample processing apparatus. By pressing the sample container 1, the fluid outlet port 15 of the sample container 1 is closely engaged with the opening 18 through the locking member 4. Subsequently, the rotating plates 21 and 22 are inserted into the grooves 9 and 10 of the sample container 1, respectively, and the rotating plates 21 and 22 are rotated so that the sample container main body 2 is rotated by 180 degrees with respect to the locking member 4. The rotation stopper release bar 23 is also rotated along with the rotating plates 21 and 22. FIGS. 3A and 3B show the state after rotation with respect to axis A. Since the sample container 1 is rotated, the fluid outlet port 15 can communicate with the opening 18 through the channel 7 provided in the locking member 4.

When a negative pressure is generated in the micro-fluidic device 16 communicating with the opening 18, the sample fluid Sa is transferred from the reservoir 3 through the channels 6, 7, and 19 to an analysis processing portion B in the micro-fluidic device 16. The transferred sample fluid is subjected to various analysis processes in the micro-fluidic device 16. Examples of the analysis processes include extraction of nucleic acid, such as DNA, contained in the sample fluid, and composition analyses of substances contained in the sample fluid.

As described above, after the fluid outlet port 15 of the sample container 1 is closely engaged with the opening 18 through the locking member 4, the locking member 4 moves relative to the sample container main body 2, and the fluid outlet port 15 is opened. Consequently, it is possible to reduce the possibility that the sample fluid Sa scatters to the outside of the channel 19 as well as the possibility that the sample fluid Sa is contaminated by the air. Furthermore, it is not necessary to thrust a needle or the like into the plug. Since a pipette is not used, a pipette chip is also not required.

The user of the sample processing apparatus of the present invention can dispose of the sample container 1 after the transfer of the sample fluid. Specifically, the rotating plates 21 and 22 and the device retainer 20 are detached from the sample container 1, and the pressure from the upper side to the sample container 1 is released. The sample container 1 and the micro-fluidic device 16 can be removed in an integrated manner and disposed of. Since the sample container 1 and the micro-fluidic device 16 can be disposed of in an integrated manner, it is possible to further reduce the possibility that the sample fluid scatters to the outside of the sample container 1. It is of course possible to perform analyses a plurality of times using one micro-fluidic device 16.

In such a case, by bringing the rotating plates 21 and 22 back to the original positions, the channels 6 and 7 are disconnected from each other. Then, only the sample container 1 is removed and disposed of. After washing the inside of the micro-fluidic device by an appropriate washing process, the micro-fluidic device may be used again in an analysis process.

### Second Embodiment

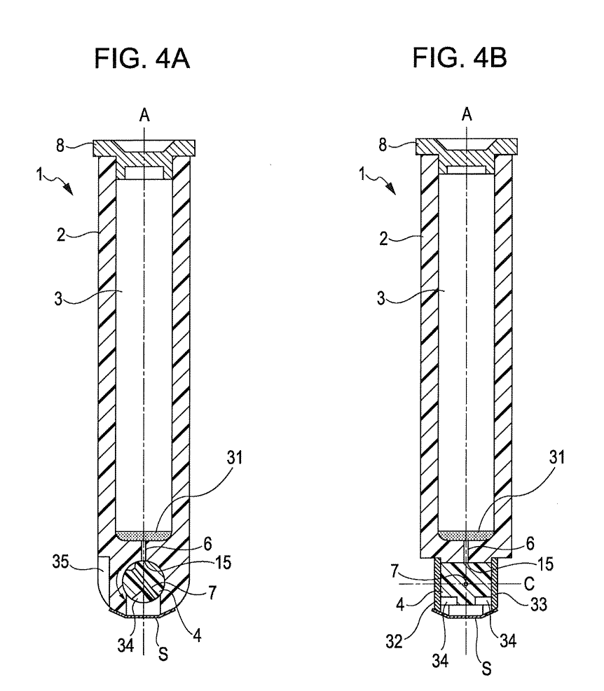

FIGS. 4A and 4B are schematic diagrams showing a sample container that can be used in a sample processing apparatus according to a second embodiment of the present invention. FIG. 4A is a cross-sectional view taken along a plane including a groove 35 provided on the side face of a sample container 1 and a locking member 4. FIG. 4B is a cross-sectional view of the sample container taken along a plane rotated from the cross section of FIG. 4A by 90 degrees with respect to axis A as in FIG. 1B. The same components as those in FIGS. 1A and 1B according to the first embodiment are represented by the same reference numerals.

When blood is used as a sample fluid, as shown in FIGS. 4A and 4B, as an anticoagulant 31, ethylene-diamine-tetraacetic acid (EDTA) may be placed in a reservoir 3. If necessary, this can be used by the user in any of the embodiments of the present invention.

A cylindrical locking member 4 is fitted in a rotatable manner in a horizontal hole provided in a sample container main body 2, and as shown in FIG. 4B, retainers 32 and 33 are joined to both sides. By rotating the cylindrical locking member 4 with respect to axis C, a fluid outlet port 15 is allowed to communicate with a channel 7 provided in the locking member 4. When a sample fluid is stored in a reservoir 3, first, the fluid outlet port 15 is closed by rotating the locking member 4 by a predetermined angle with respect to the sample container main body 2. Then, the sample fluid is stored in the reservoir 3.

A recess 34 is provided in the locking member 4, and the groove 35 is provided in the sample container main body 2. The locking member 4 is composed of a synthetic resin having good slidability, such as nylon, polyacetal, polypropylene, or a fluorocarbon resin.

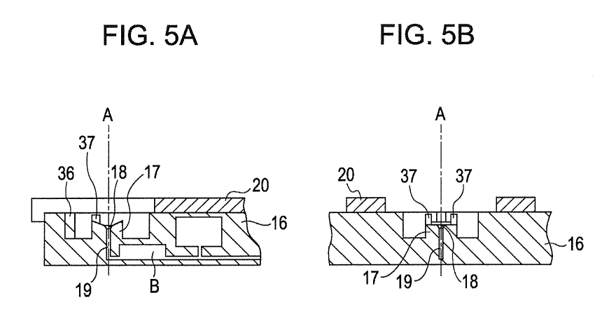

A method for transferring a sample fluid, such as blood, from the sample container 1 to a sample processing apparatus according to this embodiment will be described below with reference to FIGS. 5A and 5B. FIG. 5A is a cross-sectional view taken along a plane including an opening 18 of the sample processing apparatus and a channel 19 communicating with the opening 18, as in FIG. 2A. FIG. 5B is a cross-sectional view of the sample processing apparatus taken along a plane rotated from the cross section of FIG. 5A by 90 degrees with respect to axis A.

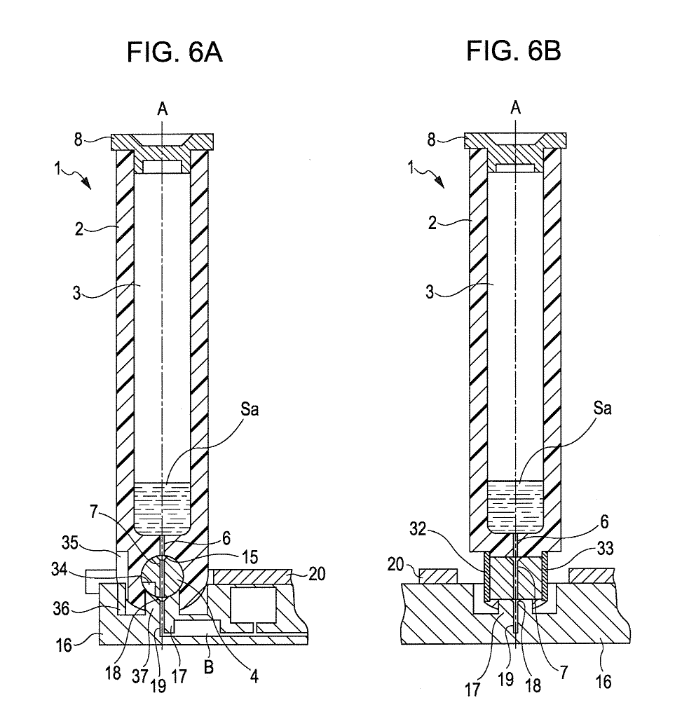

FIGS. 6A and 6B are schematic diagrams showing a micro-fluidic device 16 as an example in this embodiment. FIG. 6A is a cross-sectional view taken along a plane including the opening 18 of the sample processing apparatus and the channel 19 communicating with the opening 18, as in FIG. 3A. FIG. 6B is a cross-sectional view of the micro-fluidic device taken along a plane rotated from the cross section of FIG. 6A by 90 degrees with respect to axis A. The same components as those described in the previous embodiment are represented by the same reference numerals.

Referring to FIGS. 5A and 5B, a projection 36 and a projection 37 are provided in a sample container receiving portion 17. After the user removes the protective seal S, the sample container 1 is connected to the sample container receiving portion 17 from the upper side of the micro-fluidic device 16 such that the groove 35 faces the projection 36.

As shown in FIGS. 6A and 6B, the sample container 1 is connected to the micro-fluidic device 16 along axis A as an engaging axis. Simultaneously with the connection, the projection 37 provided on the sample container receiving portion 17 is engaged with the recess 34 provided in the locking member 4, and the locking member 4 is rotated. As a result, as shown in FIGS. 6A and 6B, the fluid outlet port 15 is closely engaged with the opening 18 through the locking member 4. Thus, the locking member 4 moves with respect to the sample container 1, and the fluid outlet port 15 communicates with the channel 7 and the opening 18. The sample container 1 is pressed from the upper side by a mechanism (not shown) of the sample processing apparatus and connected to the micro-fluidic device. Alternatively, pressing may be performed manually. When a negative pressure is generated in the micro-fluidic device 16 communicating with the opening 18, the sample fluid Sa is transferred from the reservoir 3 through the channels 6, 7, and 19 to an analysis processing portion B in the micro-fluidic device 16.

The transferred sample fluid is subjected to various analysis processes in the micro-fluidic device. Examples of the analysis processes include extraction of DNA contained in the sample fluid and composition analysis of the sample fluid.

In this embodiment, the channels 6, 7, and 19 are allowed to communicate with each other only by pressing the sample container 1 into the sample container receiving portion 17. Consequently, the structure of the sample processing apparatus can be further simplified.

After completion of the analysis processes, the device retainer 20 is detached from the sample container 1, and the pressure from the upper side to the sample container 1 is released. The user can remove and dispose of the sample container 1 and the micro-fluidic device 16 from the sample processing apparatus in an integrated manner. As described in the first embodiment, only the sample container 1 may be disposed of, and the micro-fluidic device may be reused after appropriate washing.

Furthermore, when blood is used as the sample fluid, by storing the anticoagulant 31 in the reservoir 3, it is possible to perform an anticoagulation process immediately after blood collection. In addition to EDTA, heparin or sodium citrate may be used as the anticoagulant 31. These anticoagulants may be used appropriately. If not required, the anticoagulant need not be used.

Furthermore, as necessary, in addition to the anticoagulant, a cytolytic agent or a hemolytic agent may be stored in order to process the sample blood. Alternatively, centrifugal separation may be performed. In either case, the number of processes required in the micro-fluidic device 16 can be decreased, and the total analysis time can be reduced.

### Third Embodiment

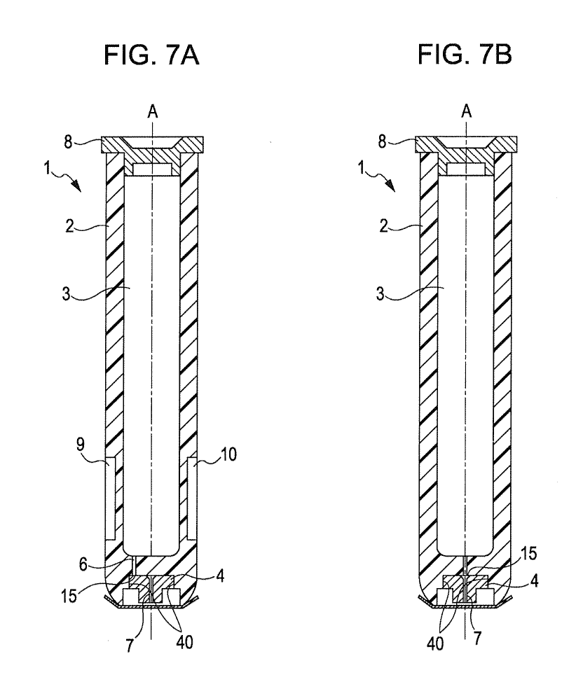

FIGS. 7A and 7B are schematic diagrams showing a sample container that can be used in a sample processing apparatus according to a third embodiment of the present invention. FIG. 7A is a cross-sectional view taken along a plane including grooves 9 and 10 provided on the side face of a sample container 1 and a locking member 4. FIG. 7B is a cross-sectional view of the sample container taken along a plane rotated from the cross section of FIG. 7A by 90 degrees with respect to axis A as in FIG. 1B. The same components as those described in the previous embodiments are represented by the same reference numerals.

Referring to FIGS. 7A and 7B, a screw portion 40, which is provided on the upper periphery of the locking member 4, is screwed into a screw hole provided in a sample container main body 2. As shown in FIGS. 7A and 7B, by turning the screw in close contact with the sample container main body 2, the locking member 4 is either brought into close contact with the sample container main body 2 so as to cover a fluid outlet port 15, or detached from the sample container main body 2. By turning the screw, the fluid outlet port 15 can be opened or closed. While a channel 7 is located at the center of the sample container main body 2, a channel 6 is eccentric to the center of the sample container main body 2. Therefore, as long as the locking member 4 is in close contact with the sample container main body 2, the channel 6 and the channel 7 do not communicate with each other.

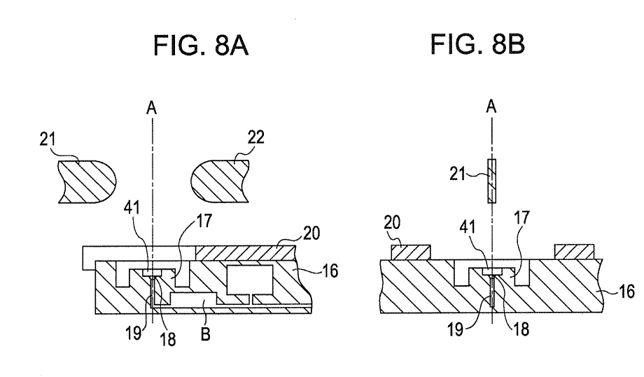

FIGS. 8A and 8B are schematic diagrams showing a part of a micro-fluidic device to which a sample fluid, such as blood, is transferred from the sample container 1 according to the third embodiment, and a part of a sample processing apparatus in which the sample is examined using the micro-fluidic device. FIG. 8A is a cross-sectional view taken along a plane including an opening 18 of the sample processing apparatus and a channel 19 communicating with the opening 18. FIG. 8B is a cross-sectional view of the sample processing apparatus taken along a plane rotated from the cross section of FIG. 8A by 90 degrees with respect to axis A. A recess 41 that engages with the lower part of the locking member 4 is disposed in a sample container receiving portion 17. For example, the lower part of the locking member 4 may be formed so as to have an equilateral triangular cross-sectional shape, and the sample container receiving portion 17 may be provided with an equilateral triangular recess that engages with the shape of the lower part of the locking member 4. Of course, the cross-sectional shapes thereof are not particularly limited as long as they can be engaged with each other.

Furthermore, as in the first embodiment, a device retainer 20, which fixes the micro-fluidic device 16, and rotating plates 21 and 22, which are inserted into the grooves 9 and 10 of the sample container main body 2, respectively, are disposed on the apparatus in which the sample is examined. The locking member 4 is composed of a synthetic resin having good slidability, such as nylon, polyacetal, polypropylene, or a fluorocarbon resin.

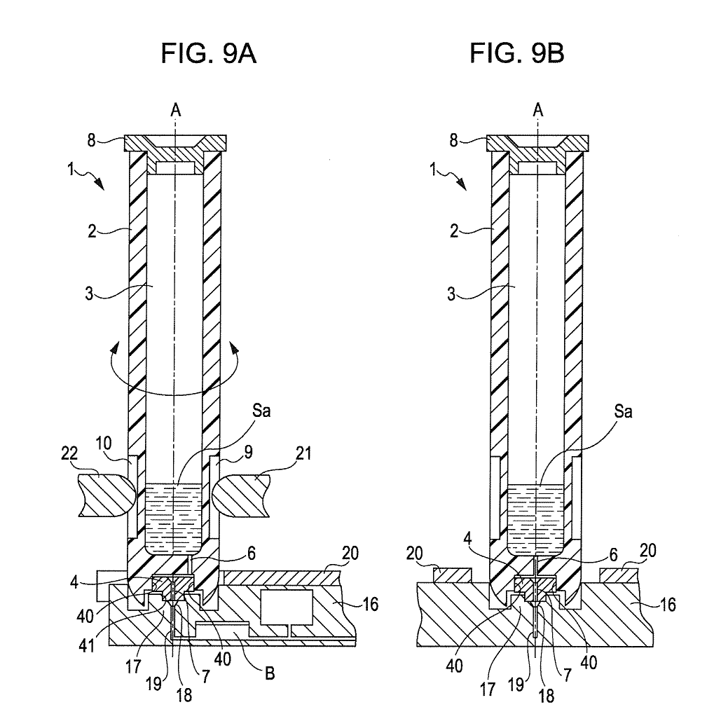

FIG. 9A is a cross-sectional view taken along a plane including the opening 18 of the sample processing apparatus and the channel 19 communicating with the opening 18. FIG. 9B is a cross-sectional view of the sample container taken along a plane rotated from the cross section of FIG. 9A by 90 degrees with respect to axis A.

After the protective seal S is removed from the sample container 1, the sample container 1 is engaged with the sample container receiving portion 17 from above the micro-fluidic device 16 such that the lower part of the locking member 4 and the recess 41 face each other. Thereby, the channel 7 and the opening 18 communicate with each other. The sample container 1 is pressed from the upper side by a mechanism (not shown) of the sample processing apparatus, and the locking member 4 and the sample container receiving portion 17 are brought into close contact with each other. Subsequently, the rotating plates 21 and 22 are inserted into the grooves 9 and 10, respectively, and the rotating plates 21 and 22 are rotated so that the sample container main body 2 is rotated with respect to the locking member 4. As a result, the locking member 4 is detached from the sample container main body 2, and as shown in FIGS. 9A and 9B, the channel 6 communicates with the channel 7 and the channel 19. Since the screw hole provided in the sample container main body 2 is coated with a sealing material, the sample fluid is prevented from leaking out of the screw hole. When a negative pressure is generated in the micro-fluidic device 16 communicating with the opening 18, the sample fluid Sa is transferred from the reservoir 3 through the channels 6, 7, and 19 to an analysis processing portion B in the micro-fluidic device 16. The sample processing apparatus performs subsequent analysis processes. Examples of the analysis processes include extraction of DNA contained in the sample fluid and composition analysis of the sample fluid.

After completion of the analysis processes, the rotating plates 21 and 22 and the device retainer 20 are detached from the sample container 1, and the pressure to the sample container 1 is released. The user of the sample processing apparatus of the present invention can remove and dispose of the sample container 1 and the micro-fluidic device 16 in an integrated manner from the sample processing apparatus. As in the previous embodiments, the micro-fluidic device can be reused.

In all the embodiments described above, collected blood, cerebrospinal fluid, or the like may be used as the sample fluid. Furthermore, the embodiments are applicable to a sample container containing dispensed blood and a specimen of DNA extracted and purified from blood. In addition to blood, examples of the sample fluid that can be used include, but are not limited to, a sample fluid obtained from treated sputum, stool, mucosa, tissues, or the like, urine, and other various fluids.

An example of the sample container is a container having a structure in which the locking member described above is provided on the bottom of a commercially available vacuum blood-collecting tube. Such a structure preferably has a highly airtight mechanism so that the reduced pressure state in the blood-collecting tube can be maintained when the container is locked by the locking member.

Furthermore, although the sample container 1 is provided with the plug 8 on the top thereof in each of the embodiments, a sample container 1 without a plug 8 may be used. In such a case, although the sample fluid is in contact with the air, it is possible to reduce the scattering of the sample fluid to the outside of the sample container 1 when the sample fluid is transferred.

Furthermore, although the example has been described in which the sample fluid, such as blood, is directly transferred to the micro-fluidic device, the sample fluid may be indirectly transferred through another component to the micro-fluidic device, or the sample fluid may be transferred to a container or an apparatus other than the micro-fluidic device.

Furthermore, although the sample container 1 is connected to the micro-fluidic device 16 by the user in each of the embodiments, the sample processing apparatus may be configured to automatically perform connection.

Furthermore, although the protective seal S is removed in each of the embodiments, in the case of using a protective seal made of a material or having a structure in which the channel 7 or 19 is not blocked even if the seal is broken, or in the case of a sample container receiving portion 17 having a shape that allows the seal to be broken so as not to block the channel, the sample container 1 may be pushed in the sample container receiving portion 17 without removing the seal.

The present invention is not limited to the above embodiments and various changes and modifications can be made within the spirit and scope of the present invention. Therefore to apprise the public of the scope of the present invention, the following claims are made.

This application claims the benefit of Japanese Application No. 2007-042660 filed Feb. 22, 2007, which is hereby incorporated by reference herein in its entirety.

What is claimed is:

1. A sample processing apparatus comprising:
a sample container having a reservoir that stores a sample fluid, a fluid outlet port provided on said reservoir, and a locking member capable of opening and closing said fluid outlet port;
a sample container receiving portion having an opening and engageable with said locking member; and
an analysis processing portion that communicates with said opening,
wherein, when said locking member of the sample container closely engages with said sample container receiving portion and said locking member moves relative to said sample container, said fluid outlet port is opened, and the sample fluid stored in said reservoir moves through said opening to said analysis processing portion so that the sample fluid can be subjected to an analysis process.

2. The sample processing apparatus according to claim 1, wherein the analysis process is a process in which nucleic acid contained in the sample fluid is extracted.

3. A sample container comprising:
a reservoir that stores a sample fluid therein;
a fluid outlet port provided on said reservoir; and
a locking member that opens and closes said fluid outlet port,
wherein said sample container can be attached to and detached from a sample processing apparatus provided with a sample container receiving portion, and when said locking member closely engages with the sample container receiving portion and said locking member moves relative to the sample container, said fluid outlet port is opened.

4. The sample container according to claim 3, wherein said sample processing apparatus performs a process of extracting nucleic acid contained in the sample fluid.
